(19) United States
(12) Patent Application Publication
(10) Pub. No.: US 2005/0000040 A1
(43) Pub. Date: Jan. 6, 2005

(54) Use of one or several substances selected from the group of pyrimidines and purines in cosmetic preparations for coloring hair

(75) Inventors: Werner Berens, Chevy Chase, MD (US); Rainer Wolber, Hamburg (DE); Christoph Smuda, Bonningstedt (DE); Franz Stab, Echem (DE); Thomas Blatt, Wedel (DE); Kyra Sanger, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/820,869

(22) Filed: Apr. 9, 2004

Related U.S. Application Data
(63) Continuation of application No. PCT/EP02/11158, filed on Oct. 4, 2002.

(30) Foreign Application Priority Data
Oct. 12, 2001 (DE) 101 50 445.4

(51) Int. Cl. A61K 31/505; A61K 7/13

(52) U.S. Cl. 8/406

(57) ABSTRACT

Use of one or more substances selected from the group of pyrimidines and purines for intensifying the natural hair color and/or for stimulating melanogenesis in human hair.

### CROSS-REFERENCE TO RELATED APPLICATIONS

The present application is a continuation of International Application No. PCT/EP02/11158, filed Oct. 4, 2002, the entire disclosure whereof is expressly incorporated by reference herein, which claims priority under 35 U.S.C. § 119 of German Patent Application No. 101 50 445.4, filed Oct. 12, 2001.

The present invention relates to a novel use and a novel method of coloring hair, For example gray hair, in particular living human hair, with one or more substances chosen from the group of pyrimidines and purines, and to preparations which are used in carrying out the method.

Hair is the thread-like skin appendage which is virtually universal (lacking on palms of the hand, soles of the feet, extensor sides of the distal phalanges of the toes and fingers); differentiated as long hair (head hair, beard hair, axilla hair, pubic hair=capilli, barba, hirci and pubes, respectively; in men also chest hair), short, bristle hair (supercilia, cilia, vibrissae, tragi) and down (lanugo, vellus hair). The structure of all these hairs is approximately and on the whole similar: in the centre the hair medulla (comprising epithelial cells with eosinophilic horny substance granules=trichohyalin granules), surrounded by the hair cortex (comprising keratinized cells; comprises pigments) and the outer skin of the hair (cuticula pili; anuclear epidermis layer) and by layers of the epithelial and connective tissue hair sheath.

The hair is divided into the hair shaft protruding from the skin and the inclined hair root reaching into the subcutis and whose layers correspond approximately to those of the epidermis. The thickened lower root end, the hair bulb, sits on a vascular connective tissue pin, the hair papilla, protruding into it (both as hair base). The bulb in the starting (=anagen) phase of the cyclically repeating hair formation is coated onion-like as a result of the continuous new formation of cells by its near-papillary layer (matrix), then later closed, bulb-like, very keratinized (bulb hair) and is finally, in the end (=telogen) phase, displaced in the direction of the follicle opening by a new hair—starting from a newly forming hair papilla.

( . . . ) Hair formation begins in the third embryonal month as a result of epidermal sprouting into the subcutis. The lifetime is about 3 years. The terminal hairs displacing the fuzzy down are longer, stronger and darker; as head hair, eyebrows, eyelashes, they appear soon after birth, but otherwise during puberty under the influence of hormones. Functions: heat insulation (also heat dissipation as a result of enlargement of the evaporation surface for sweat), reduction of friction and—as a result of the surrounding nerves—involvement in the sense of touch.

Melanin is responsible for personal hair color. The melanin is formed in the melanocytes, cells which arise in the hair bulb associated with the keratinocytes of the hair medulla. Melanocytes contain melanosomes as characteristic cell organelles where the melanin is formed. This is transferred via the long dendrites of the melanocytes to the keratinocytes of the precortical matrix and brings about the more or less marked blond to brown-black hair color. Melanin is formed as the final stage of an oxidative process in which tyrosine converts, with the assistance of the enzyme tyrosinase, via several intermediates to the brown to brown-black eumelanins (DHICA and DHI melanin) and/or, with participation of sulfur-containing compounds, to the reddish pheomelanin. DHICA and DHI melanins arise via the common intermediate stages dopaquinone and dopachrome. The latter is converted, partially with participation of further enzymes, either into indole-5,6-quinonecarboxylic acid or into indole-5,6-quinone, from which the two specified eumelanins form. The formation of pheomelanin proceeds, inter alia, via the intermediate products dopaquinone and cysteinyldopa. Cysteine is additionally necessary when the pheomelanin is to arise for blond and reddish hair.

The eumelanin is the black-brown pigment. It primarily determines the color depth of the hair. In brown and black hair it is present in clearly visible granules.

Pheomelanin is the red pigment. It is responsible for pale blond, blond and red hair. Due to its structure, this melanin is very much finer and smaller. The various proportions of the melanin types lead to the various hair colors:
Blond hair contains a small amount of eumelanin and a large amount of pheomelanin.
Dark hair contains a large amount of eumelanin and a small amount of pheomelanin.
Red hair likewise has a small amount of eumelanin and a very large amount of pheomelanin.
All shades of hair in between result from varying mixing ratios of the two melanin types.

The pigment formation process can only proceed if sufficient tyrosinase is available. This enzyme is formed more infrequently with increasing age. This then gradually leads to gray hair. The reason: with little tyrosinase, less and less tyrosine is also formed. The production of melanin thus decreases. The lack of melanin is replaced by the inclusion of air bubbles. The hairs appear gray.

This process is usually insidious. It starts at the temples and then extends to the entire head hair. Subsequently, it affects the beard and the eyebrows. In the end, all of the hair on the body is finally gray.

In medical terms, gray hairs are referred to as canities. There are various graying possibilities. Premature graying, from age 20, is also called canities praecox.

Canities symptomatica, or symptomatic graying of the hair, can have various causes. These include:
Pernicious anemia (vitamin B deficiency anemia),
Severe endocrinological disorders, e.g. in the case of thyroid disorders,
Acute febrile illnesses,
Treatment side-effects,
Cosmetics,
Metals.

The coloring of hair, in particular of living human hair, using natural dyes, as has been known since antiquity, particularly for the dye henna, and which has been pushed into the background in favor of synthetic dyes has for some years been the object of new interest. The red shade which arises with henna is a disadvantage.

Melanin production, which brings about the hair color, decreases with increasing age: the hair becomes gray or white. It is a cosmetic wish for some consumers to reverse or to slow this process. For this purpose, the cosmetics industry in some countries uses lead acetate which is toxic and is therefore prohibited in the European Cosmetics Directive. This lead acetate is preferably applied in the form of a solution to the hair and remains there for a prolonged period without being washed off.

For the dyeing of keratin-containing fibers, e.g. hair, wool or furs, use is generally made either of direct dyes or oxidation dyes, which are formed by oxidative coupling of one or more developer components with one another or one or more coupler components. Coupler and developer components are also referred to as oxidation dye precursors.

The developer components used are usually primary aromatic amines with a further free or substituted hydroxyl or amino group, situated in the para or ortho position, diaminopyridine derivatives, heterocyclic hydrazones, 4-aminopyrazolone derivatives, and 2,4,5,6-tetraaminopyrimidine and derivatives thereof.

Specific representatives are, for example, p-phenylenediamine, p-tolylenediamine, 2,4,5,6-tetraaminopyrimidine, p-aminophenol, N,N-bis(2-hydroxyethyl)-p-phenylenediamine, 2-(2,5-diaminophenyl)ethanol, 2-(2,5-diaminophenoxy)ethanol, 1-phenyl-3-carboxyamido-4-amino-5-pyrazolone, 4-amino-3-methylphenol, 2-aminomethyl-4-aminophenol, 2-hydroxymethyl-4-aminophenol, 2-hydroxy-4,5,6-triaminopyrimidine, 2,4-dihydroxy-5,6-diaminopyrimidine and 2,5,6-triamino-4-hydroxypyrimidine.

Coupler components used are usally m-phenylenediamine derivatives, naphthols, resorcinol and resorcinol derivatives, pyrazolones and m-aminophenols. Suitable couplers substances are, in particular, α-napthol, 1,5-, 2,7-, 1,7-dihydroxynaphthalene, 5-amino-2-methylphenol, m-aminophenol, resorcinol, resorcinol monomethyl ether, m-phenylenediamine, 2,4-diaminophenoxyethanol, 1-phenyl-3-methyl-5-pyrazolone, 2,4-dichloro-3-aminophenol, 1,3-bis(2,4-diaminophenoxy)propane, 2-chlororesorcinol, 4-chlororesorcinol, 2-chloro-6-methyl-3-aminophenol, 2-methylresorcinol and 5-methylresorcinol.

With regard to further customary dye components, reference is made expressly to the series “Dermatology”, published by Ch. Culnan, H. Maibach, Verlag Marcel Dekker Inc., New York, Basel, 1985, Vol. 7, Ch. Zviak, The Science of Hair Care, Ch. 7, pages 248-250 (Direct Dyes), and Ch. 8, pages 264-267 (Oxidation Dyes), and also the “European inventory of cosmetic raw materials”, 1996, published by the European Commission, obtainable in diskette form from the Bundesverband der deutschen Industrie- und Handelsunternehmen für Arzneimittel, Reformwaren und Körperpflegemittel e.V., Mannheim.

Although intensive colorations with good fastness properties can be achieved with oxidation dyes, the development of the color generally takes place under the influence of oxidizing agents, such as, for example, H₂O₂, which in some cases can result in damage to the fibers. Furthermore, some oxidation dye precursors or certain mixtures of oxidation dye precursors can occasionally have a sensitizing effect in people with sensitive skin. Although direct dyes are applied under more moderate conditions, their disadvantage is that the colorations frequently have only inadequate fastness properties.

It would be desirable to have available colorants for keratin fibers, in particular human hair, which, with regard to depth of color, gray coverage and fastness properties, are at least equal in qualitative terms to otherwise customary oxidation hair dyes, without necessarily having to use oxidizing agents, such as, for example, H₂O₂. It would also be desirable for the colorants to have no or only a very low sensitizing potential.

It was surprising and could not have been foreseen by the person skilled in the art that the use of one or more substances chosen from the group of pyrimidines and purines for boosting natural skin tanning and/or for stimulating melanogenesis in human skin may overcome the disadvantages of the prior art.

A particular advantage is that, as a result of the present invention, physiological processes (increased synthesis of melanin) of the skin and of the hair are utilized in order to obtain the desired pigmentation of the hair, and thus the intensification of the hair color. Purines represent a group of important compounds which are widespread in nature and participate in human, animal, plant and microbial metabolic processes, and which derive from the parent substance purine through substitution by OH, NH₂, SH in the 2, 6 and 8 position and/or by CH₃ in the 1, 3, 7 position.

;The basic framework of purine and its derivatives is characterized by the following structure:

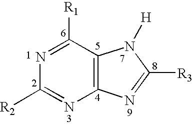

| | R₁ | R₂ | R₃ |
|---|---|---|---|
| Purine | H | H | H |
| Adenine | NH₂ | H | H |
| Guanine | OH | NH₂ | H |
| Uric acid | OH | OH | OH |
| Hypoxanthine | OH | H | H |
| Purinethiol | SH | H | H |
| 6-Thioguanine | SH | NH₂ | H |
| 6-Xanthine | OH | OH | H |

The biosynthesis of the purine takes place at the nucleotide stage from glycine and CO₂, and small molecular fragments of L-glutamine, of L-aspartic acid and of 10-formyltetrahydrofolic acid. In metabolism, purine bases are released which are partly reutilized in the cells, i.e. converted into one another.

The most important purines include adenine and guanine which—together with the pyrimidines uracil, thymine and cytosine—are constituents of nucleic acids, also hypoxanthine, xanthine and uric acid as metabolic products of humans and animals, and the plant purines, often referred to as purine alkaloids, caffeine, theobromine and theophylline, which are present in coffee, cocoa and tea.

Plant growth substances which likewise belong to the purines are zeatin and kinetin (cytokinins). Among the animal foodstuffs, the innards, particularly thymus, are rich in purines, and fish and green peas also contain relatively large amounts.

For the purposes of the present invention, pyrimidines are the derivatives of pyrimidine

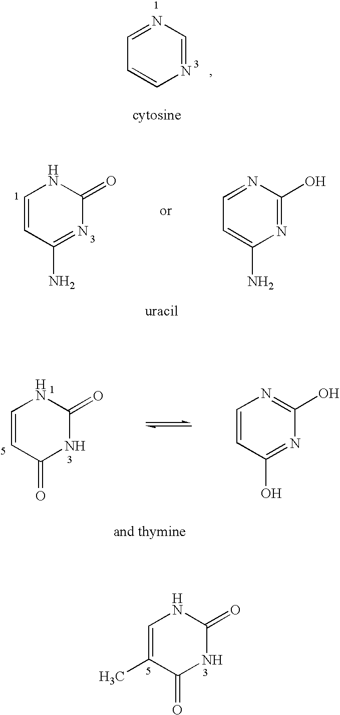

Cytosine and thymine are counterparts of adenine and guanine in deoxyribonucleic acid. The role of cytosine is taken over by uracil in ribonucleic acid.

Surprisingly, it has been found that one or more substances chosen from the group of pyrimidines and purines increase the activity of the melanocytes of the human skin and hair and thus also melanogenesis as a physiological process and as a result enhance natural pigmentation of the hair. This leads to the intensification of the (natural) hair color.

Preparations according to the invention advantageously comprise 0.0001-20 percent by weight of one or more substances chosen from the group of pyrimidines and purines, preferably purine and/or uracil and/or thymine and/or adenine and/or guanine and/or cytosine.

Preparations according to the invention preferably comprise 0.001-10% by weight of one or more substances chosen from the group of pyrimidines and purines, preferably purine and/or uracil and/or thymine and/or adenine and/or guanine and/or cytosine, based on the total composition of the preparations.

Preparations according to the invention very particularly preferably comprise 0.01-1% by weight of one or more substances chosen from the group of pyrimidines and purines, preferably purine and/or uracil and/or thymine and/or adenine and/or guanine and/or cytosine, based on the total composition of the preparations.

According to the invention the cosmetic and/or dermatological preparations can have the customary composition.

For use, the cosmetic and dermatological preparations according to the invention are applied to the scalp and/or hair in sufficient amount and in the manner conventional for cosmetics.

According to the invention the cosmetic and dermatological preparations according to the invention can comprise cosmetic auxiliaries such as those conventionally used in such preparations, e.g. preservatives, bactericides, perfumes, antifoams, dyes, pigments which have a coloring effect, thickeners, moisturizers and/or humectants, fats, oils, waxes or other conventional constituents of a cosmetic or dermatological formulation, such as alcohols, polyols, polymers, foam stabilizers, electrolytes, organic solvents or silicone derivatives.

An additional content of antioxidants is generally preferred. According to the invention, favorable antioxidants which can be used are any antioxidants suitable or customary for cosmetic and/or dermatological applications.

It is also advantageous to add antioxidants to the preparations according to the invention. The antioxidants are advantageously selected from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximines) in very low tolerated doses (e.g. pmol to pmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of the abovementioned antioxidants (one or more compounds) in the preparations according to the invention, is preferably from 0.001 to 30% by weight, particularly preferably from 0.05 to 20% by weight, especially 1-10% by weight, based on the total weight of the preparation.

If vitamin E and/or derivatives thereof are used as the antioxidant or antioxidants, their respective concentrations are advantageously chosen from the range of 0.001-10% by weight, based on the total weight of the formulation.

If vitamin A or vitamin A derivatives or carotenes or derivatives thereof are used as the antioxidant or antioxidants, their respective concentrations are advantageously chosen from the range of 0.001-10% by weight, based on the total weight of the formulation.

The lipid phase can advantageously be chosen from the following group of substances:
mineral oils, mineral waxes
oils, such as triglycerides of capric or caprylic acid, but preferably castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alkyl benzoates;
silicone oils such as dimethylpolysiloxanes, diethylpolysiloxanes, diphenylpolysiloxanes and mixtures thereof.

For the purposes of the present invention, the oil phase of the emulsions, oleogels and hydrodispersions or lipodispersions is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, from the group of saturated or unsaturated, branched or unbranched alcohols, and also fatty acid triglycerides, namely the triglycerol esters of saturated and/or unsaturated, branched and/or unbranched alkanecarboxylic acids having a chain length of from 8 to 24, in particular 12-18, carbon atoms. The fatty acid triglycerides can advantageously be chosen, for example, from the group of synthetic, semisynthetic and natural oils, e.g. olive oil, sunflower oil, soybean oil, peanut oil, rapeseed oil, almond oil, palm oil, coconut oil, palm kernel oil and the like.

For the purposes of the present invention, any mixtures of such oil and wax components can also advantageously be used. When required, it can also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene are advantageously to be used for the purposes of the present invention.

The oil phase can advantageously also contain cyclic or linear silicone oils or can consist entirely of such oils, although it is preferable to use an additional content of other oil phase components in addition to the silicone oil or silicone oils.

Cyclomethicone (octamethylcyclotetrasiloxane) is advantageously used as the silicone oil to be used according to the invention. However, other silicone oils can also advantageously be used for the purposes of the present invention, for example hexamethylcyclotrisiloxane, polydimethylsiloxane, poly(methylphenylsiloxane).

Mixtures of cyclomethicone and isotridecyl isononanoate and mixtures of cyclomethicone and 2-ethylhexyl isostearate are also particularly advantageous.

The aqueous phase of the preparations according to the invention may advantageously comprise
alcohols, diols or polyols of low carbon number, and also their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and especially one or more thickeners which can advantageously be selected from the group consisting of silicon dioxide, aluminum silicates and polysaccharides and derivatives thereof, e.g. hyaluronic acid, xanthan gum, hydroxypropylmethylcellulose, and particularly advantageously from the group of polyacrylates, preferably a polyacrylate from the group consisting of the so-called Carbopols, for example Carbopol grades 980, 981, 1382, 2984, 5984, in each case individually or in combination.

The examples below serve to illustrate the present invention without limiting it. Unless stated otherwise, all amounts, proportions and percentages are based on the weight and the total amount or on the total weight of the preparations.

| | % by wt. |
|---|---|
| Example 1: Spray formulation | |
| Ethanol | 28.00 |
| Uracil | 0.80 |
| Ubiquinone 10 | 0.10 |
| Preservative, dyes, perfume | q.s. |
| Propane/butane 25/75 | ad 100.00 |
| Example 2: Shower bath | |
| Sodium laureth sulfate | 33.00 |
| Potassium cocoyl hydrolyzed collagen (30%) | 11.00 |
| Cocoamphodiacetate (30%) | 5.00 |
| PEG-7 glyceryl cocoate | 2.00 |
| Cocamide MEA | 1.00 |
| Sodium chloride | 0.50 |
| Uracil | 0.50 |
| Citric acid | 0.02 |
| Preservative, dyes, perfume | q.s. |
| Water | ad 100.00 |
| Example 3: Conditioning | |
| shampoo with pearlescence | |
| Polyquaternium-10 | 0.50 |
| Sodium laureth sulfate | 9.00 |
| Cocoamidopropylbetaine | 2.50 |
| Pearlizing agent | 2.00 |
| Uracil | 0.50 |
| Preservative, perfume, thickener, | q.s. |
| pH adjustment and | |
| solubility promoter | |
| Water | ad 100.00 |
| The pH is adjusted to 6. | |
| Example 4: Clear conditioning shampoo | |
| Polyquaternium-10 | 0.50 |
| Sodium laureth sulfate | 9.00 |
| Cocoamidopropylbetaine | 2.50 |
| Uracil | 0.50 |
| Folic acid | 0.20 |
| Preservative, perfume, thickener, | q.s. |
| pH adjustment and | |
| solubility promoter | |
| Water | ad 100.00 |
| The pH is adjusted to 6. | |
| Example 5: Clear light | |
| shampoo with volume effect | |
| Sodium laureth sulfate | 10.00 |
| Cocoamidopropylbetaine | 2.50 |
| Uracil | 0.50 |
| Preservative, perfume, thickener, | q.s. |
| pH adjustment and | |
| solubility promoter | |
| Water | ad 100.00 |
| The pH is adjusted to 5.5. | |
| Example 6: Hair treatment | |
| Hydroxypropylmethylcellulose | 0.50 |
| Cetrimonium bromide | 1.00 |
| Glycerol | 3.00 |
| Cetearyl alcohol | 2.50 |
| Glyceryl stearate | 2.00 |
| Uracil | 0.80 |
| Preservative, perfume, pH adjustment | q.s. |
| Water | ad 100.00 |
| The pH is adjusted to 3.5. | |
| Example 7: Hair rinse | |
| Behentrimonium chloride | 1.00 |
| Glyceryl | 3.00 |
| Hydroxyethylcellulose | 0.20 |
| Cetearyl alcohol | 3.00 |
| Uracil | 1.00 |
| Folic acid | 0.80 |
| Preservative, perfume, pH adjustment | q.s. |
| Water | ad 100.00 |
| The pH is adjusted to 3.0. | |

1.-2. (canceled)

3. A composition for at least one of intensifying natural hair color and stimulating melanogenesis in human hair, wherein the composition comprises from 0.0001% to 20% by weight of at least one substance selected from pyrimidines and purines.

4. The composition of claim 3, wherein the at least one substance is present in a concentration of from 0.001% to 10% by weight.

5. The composition of claim 3, wherein the at least one substance is present in a concentration of from 0.01% to 1% by weight.

6. The composition of claim 3, wherein the composition comprises at least one of purine, uracil, thymine, adenine, guanine and cytosine.

7. The composition of claim 6, wherein the composition comprises purine.

8. The composition of claim 6, wherein the composition comprises uracil.

9. The composition of claim 6, wherein the composition comprises thymine.

10. The composition of claim 6, wherein the composition comprises adenine.

11. The composition of claim 6, wherein the composition comprises guanine.

12. The composition of claim 6, wherein the composition comprises cytosine.

13. The composition of claim 5, wherein the composition comprises uracil.

14. The composition of claim 13, wherein the composition comprises at least 0.5% by weight of uracil.

15. The composition of claim 3, wherein the composition further comprises from 0.05% to 10% by weight of an antioxidant.

16. A spray formulation which comprises the composition of claim 3.

17. A shower bath which comprises the composition of claim 3.

18. A shampoo which comprises the composition of claim 3.

19. A conditioning shampoo which comprises the composition of claim 3.

20. A hair treatment which comprises the composition of claim 3.

21. A hair rinse which comprises the composition of claim 3.

22. A composition for at least one of intensifying natural hair color and stimulating melanogenesis in human hair, wherein the composition comprises from 0.1% to 1% by weight of at least one of purine, uracil, thymine, adenine, guanine and cytosine.

23. The composition of claim 22, wherein the composition comprises uracil.

24. A method for at least one of intensifying natural hair color and stimulating melanogenesis in human hair, wherein the method comprises applying onto at least parts of the hair a composition which comprises at least one substance selected from pyrimidines and purines.

25. The method of claim 24, wherein the at least one substance is present in a concentration of from 0.0001% to 20% by weight.

26. The method of claim 24, wherein the at least one substance is present in a concentration of from 0.001% to 10% by weight.

27. The method of claim 24, wherein the at least one substance is present in a concentration of from 0.01% to 1% by weight.

28. The method of claim 24, wherein the composition comprises at least one of purine, uracil, thymine, adenine, guanine and cytosine.

29. The method of claim 25, wherein the composition comprises purine.

30. The method of claim 25, wherein the composition comprises uracil.

31. The method of claim 25, wherein the composition comprises thymine.

32. The method of claim 25, wherein the composition comprises adenine.

33. The method of claim 25, wherein the composition comprises guanine.

34. The method of claim 25, wherein the composition comprises cytosine.

35. The method of claim 24, wherein the composition comprises at least 0.5% by weight of uracil.

36. The method of claim 24, wherein the composition comprises a spray formulation.

37. The method of claim 24, wherein the composition comprises a shower bath.

38. The method of claim 24, wherein the composition comprises a shampoo.

39. The method of claim 24, wherein the composition comprises a hair treatment.

40. The method of claim 24, wherein the composition comprises a hair rinse.
